(19) United States
(12) Patent Application Publication
(10) Pub. No.: US 2021/0205247 A1
(43) Pub. Date: Jul. 8, 2021

(54) SUSTAINED-RELEASE FORMULATION CONTAINING PREGABALIN

(71) Applicant: UNI PHARMA CO., LTD., Taipei City (TW)

(72) Inventors: Chih-Hung LI, Taipei City (TW); Chen-Ming HUANG, Taipei City (TW); Li-Chuan CHUANG, Taipei City (TW)

(21) Appl. No.: 16/832,071

(22) Filed: Mar. 27, 2020

(30) Foreign Application Priority Data
Jan. 7, 2020 (TW) 109100420

(51) Int. Cl. A61K 31/197; A61K 9/00; A61K 47/36; A61K 47/38; A61K 47/32; A61K 47/12

(52) U.S. Cl. CPC A61K 31/197; A61K 9/0053; A61K 47/12; A61K 47/38; A61K 47/32; A61K 47/36

(57) ABSTRACT

Disclosed herein is a sustained-release formulation that includes, based on the total weight of the sustained-release formulation, 5 wt % to 40 wt % of pregabalin, or a pharmaceutically acceptable salt, solvate or hydrate thereof, 0.1 wt % to 5 wt % of carbomer, and 20 wt % to 60 wt % of polyethylene oxide, wherein the formulation is free from polyvinyl acetate. The formulation can release pregabalin consistently over a time period of 24 hours, and is suitable for once-daily administration.

### CROSS-REFERENCE TO RELATED APPLICATION

This application claims priority of Taiwanese Patent Application No. 109100420, filed on Jan. 7, 2020.

### FIELD

The present disclosure relates to a sustained-release formulation including pregabalin, carbomer, and polyethylene oxide, wherein the formulation is free from polyvinyl acetate. In particular, the formulation can release pregabalin consistently over a time period of 24 hours, and is suitable for once-daily administration.

### BACKGROUND

Pregabalin (chemical name: (S)−(+)-3-aminomethyl-5-methylhexanoic acid) is an analog of gamma-aminobutyric acid (GABA) which selectively binds to alpha-2-delta (α2-δ) subunits of presynaptic calcium channels of the central nerve system to reduce calcium ion influx at nerve endings, resulting in reduction of the release of several excitatory neurotransmitters such as glutamate and noradrenalin, which restores the function of nerve cells to normal levels.

Pregabalin exhibits anti-seizure activity, and has been developed as a useful drug for the treatment of various neurological diseases, such as neuropathic pain, epilepsy, fibromyalgia syndrome, Huntington's chorea, cerebral ischemia, and Parkinson's disease. Pregabalin is currently available as immediate-release formulation in the form of hard capsule shell (e.g., Lyrica®, Pfizer Inc.), and is administered to patients twice or three times daily. However, such method of administration causes considerably uncomfortable feeling to patients, and in particular, greatly decreases drug compliance of patients who are required to take pregabalin for a long time period or a large quantity of pregabalin altogether. Therefore, researchers in this field have attempted to develop new sustained-release formulations of pregabalin for once-daily administration.

U.S. 9,144,559 B2 discloses a pharmaceutical composition suitable for once-daily administration including pregabalin, a matrix forming agent, and a swelling agent, wherein the matrix forming agent includes polyvinyl acetate (PVAc) and polyvinylpyrrolidone (PVP), and the swelling agent includes crosslinked polyvinylpyrrolidone (PVPP). For solid dosage forms (e.g., tablets) administered orally, the matrix forming agent, among other functions, imparts structural integrity and helps to control or extend the rate of drug release. In addition to the matrix forming agent and the swelling agent, the pharmaceutical composition may optionally include a gelling agent, which extends the rate of drug release of the dosage form. The gelling agents include carbomers (e.g., carbopol), polysaccharides (e.g., hydroxyethyl cellulose), and combinations thereof. In the Examples section of the aforesaid patent publication, the drug product of Example 24 which contains Carbopol®71G as a gelling agent and the drug product of Example 25 which contains Natrosolr®250 as a gelling agent were subjected to a dissolution test for a time period of 24 hours. The results reveal that at the end of the 20th hour of the dissolution test, the pregabalin dissolution rate of the drug product for Example 24 was 102.7%, while at the end of the 24th hour of the dissolution test, the pregabalin dissolution rate of the drug product for Example 25 was only 99.3%.

In view of the aforesaid, there is still a need for those in the art to develop new sustained-release formulations of pregabalin for once-daily administration.

### SUMMARY

Accordingly, the present disclosure provides a sustained-release formulation including, based on the total weight of the sustained-release formulation:

5 wt % to 40 wt % of pregabalin, or a pharmaceutically

5 wt % to 40 wt % of pregabalin, or a pharmaceutically acceptable salt, solvate or hydrate thereof;

0.1 wt % to 5 wt % of carbomer; and

20 wt % to 60 wt % of polyethylene oxide;

wherein the sustained-release formulation is free from polyvinyl acetate.

### BRIEF DESCRIPTION OF THE DRAWINGS

Other features and advantages of the disclosure will become apparent in the following detailed description of the embodiments with reference to the accompanying drawings, of which:

The Figure shows the results of a dissolution test of tablets 1 to 3 of Example 1, infra, and a commercially-available LYRICA® pregabalin capsule.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly and materials similar or equivalent to those described herein, which could be used in the practice of the present disclosure. Indeed, the present disclosure is in no way limited to the methods and materials described.

Through research, the applicant suprisingly found that a formulation including pregabalin, carbomer, and polyethylene oxide can release pregabalin consistently over a time period of 24 hours. Specifically, the formulation is free from polyvinyl acetate. The formulation is expected to be useful for once-daily administration.

Therefore, the present disclosure provides a sustained-release formulation including, based on the total weight of the formulation:

5 wt % to 40 wt % of pregabalin, or a pharmaceutically acceptable salt, solvate or hydrate thereof;

0.1 wt % to 5 wt % of carbomer; and

20 wt % to 60 wt % of polyethylene oxide;

wherein the formulation is free from polyvinyl acetate.

Examples of the carbomer suitable for use in this disclosure include, but are not limited to, Acrypol®, Acritamer®, Carbopol®, and Pemulen®.

According to the present disclosure, the carbomer is selected from the group consisting of Acrypol® 974P, Acrypol® 934P, Acrypol® 971P, and combinations thereof. In an exemplary embodiment, the carbomer is Acrypol® 974P.

As used herein, the terms “sustained-release formulation” and “controlled-release formulation” may be used interchangeably, and may refer to any pharmaceutical formulation that maintains constant levels of a drug in the patient's bloodstream by releasing the drug over an extended period of time.

In certain embodiments, the sustained-release formulation is formulated to release the active ingredient (i.e., pregabalin) gradually and predictably over a 12-hour to 24-hour time period. In an exemplary embodiment, the sustained-release formulation is formulated to release pregabalin consistently over a 24-hour time period.

According to the present disclosure, the sustained-release formulation may further include a pharmaceutically acceptable carrier, and made into a dosage form suitable for oral administration using technology well-known to those skilled in the art. Examples of the dosage form include, but are not limited to, tablet, pellet, lozenge, capsule, and the like.

Examples of the pharmaceutically acceptable carrier suitable for use in this disclosure may include, but are not limited to, solvents, buffers, emulsifiers, suspending agents, decomposers, disintegrating agents, dispersing agents, binders, antioxidants, stabilizing agents, chelating agents, diluents, gelling agents, preservatives, wetting agents, lubricants, absorption delaying agents, liposomes, and combinations thereof.

In certain embodiments, the sustained-release formulation may further include, based on the total weight of the formulation:

5 wt % to 60 wt % of a diluent;

1 wt % to 10 wt % of a binder;

0.01 wt % to 2 wt % of an antioxidant; and

0.1 wt % to 5 wt % of a lubricant.

The diluent may be selected from the group consisting of starch, microcrystalline cellulose, anhydrous dicalcium phosphate, dextrose, lactose, sucrose, mannitol, xylitol, sorbitol, and combinations thereof.

The binder may be selected from the group consisting of polyvinylpyrrolidone (PVP), copovidone, hydroxypropyl cellulose, hydroxypropylmethyl cellulose, hydroxyethyl cellulose, methylcellulose, and combinations thereof.

The antioxidant may be selected from the group consisting of butylated hydroxytoluene (BHT), butylated hydroxyanisole (BHA), ascorbic acid, propyl gallate, and combinations thereof.

The lubricant may be selected from the group consisting of stearic acid, magnesium stearate, calcium stearate, zinc stearate, glyceryl monostearate, sodium stearyl fumarate, glyceryl behenate, and combinations thereof.

In an exemplary embodiment of the present disclosure, the sustained-release formulation includes 29.5 wt % pregabalin, 0.4 wt % carbomer, 20 wt % PEO, 13.9 wt % starch, 32.5 wt % microcrystalline cellulose, 2.7 wt % PVP, 0.1 wt % BHT, and 0.9 wt % magnesium stearate, based on the total weight of the sustained-release formulation.

In an exemplary embodiment of the present disclosure, the sustained-release formulation includes 29.5 wt % pregabalin, 0.4 wt % carbomer, 40 wt % PEO, 7.9 wt % starch, 18.5 wt % microcrystalline cellulose, 2.7 wt % PVP, 0.1 wt % BHT, and 0.9% magnesium stearate, based on the total weight of the sustained-release formulation.

In an exemplary embodiment of the present disclosure, the sustained-release formulation includes 29.5 wt % pregabalin, 0.4 wt % carbomer, 60 wt % PEO, 1.9 wt % starch, 4.5 wt % microcrystalline cellulose, 2.7 wt % PVP, 0.1 wt % BHT, and 0.9 wt % magnesium stearate, based on the total weight of the sustained-release formulation.

The disclosure will be further described by way of the following examples. However, it should be understood that the following examples are solely intended for the purpose of illustration and should not be construed as limiting the disclosure in practice.

### EXAMPLES

### General Experimental Materials:

1. The ingredients used for preparing tablets containing pregabalin are listed in Table 1.

TABLE 1

| Ingredients | Sources |
|---|---|
| Pregabalin | MSN Pharmaceuticals Inc. |
| Starch (trade name: Starch | Colorcon Inc. |
| 1500 ®) | |
| Carbomer (trade name: Acrypol | Corel Pharma Chem |
| 974P) | |
| Microcrystalline cellulose | Mingtai Chemical Co., Ltd. |
| (trade name: Comprecel | |
| M101D+ ®) | |
| Polyvinylpyrrolidone (PVP) | Anhui Sunhere Pharmaceutical |
| (trade name: Povidone K-30 ®) | Excipients Co., Ltd. |
| Butylated hydroxytoluene | Spectrum Chemical |
| (BHT) | |
| Polyethylene oxide (PEO) | Dow Chemical |
| (trade name: Polyox WSR 303) | |
| Magnesium stearate | Sigma-Aldrich |

### Example 1

### Preparation of Tablet Containing Pregabalin

Three tablets containing pregabalin were prepared using the recipe shown in Table 2, and the preparation process of these tablets were described as follows.

TABLE 2

| | Tablet 1 | Tablet 2 | Tablet 3 |
|---|---|---|---|
| Ingredients | Tablet (mg) | | |
| Pregabalin | 330 | 330 | 330 |
| Starch | 156 | 88.5 | 21.6 |
| Carbomer | 5 | 5 | 5 |
| Microcrystalline | 364 | 207.5 | 50.4 |
| cellulose | | | |
| PVP | 30 | 30 | 30 |
| BHT | 1 | 1 | 1 |
| PEO | 224 | 448 | 672 |
| Magnesium stearate | 10 | 10 | 10 |
| Total Weight | 1120 | 1120 | 1120 |

Pregabalin, starch, carbomer, microcrystalline cellulose, PVP, and BHT were evenly mixed, followed by granulating the resultant mixture, so as to obtain granules containing pregabalin. The granules containing pregabalin were mixed with PEO and magnesium stearate in a mixer to obtain a final mixture, which was then pressed into tablets.

### Example 2

### Dissolution Test of Tablet Containing Pregabalin

In order to evaluate the pregabalin release profiles of tablets 1 to 3 obtained in Example 1, the following analyses were conducted. In addition, for the purpose of comparison, a commercially-available LYRICA® pregabalin capsule (75 mg/cap) (Manufacturer: Pfizer Inc.) was subjected to the same evaluation.

### Methods:

The dissolution test was conducted according to the FDA-Recommended Dissolution Methods for pregabalin oral extended release tablet. Briefly, the dissolution test was performed using a USP rotating paddle apparatus, in which 900 mL of 0.06 N—HC1 buffer solution as the dissolution medium, and at a rotation speed of 50 rpm and a dissolution temperature of 37° C. For each of tablets 1 to 3, 1.5 mL of the dissolution medium was taken as sample at time points of 0.5, 1, 2, 3, 4, 6, 8, 10, and 24 hours. For the LYRICA® pregabalin capsule, 1.5 mL of the dissolution medium was taken as sample at time points of 0, 0.16, 0.25, 0.5, and 0.75 hours.

The thus obtained samples of tablets 1 to 3 and the LYRICA® pregabalin capsule were subjected to centrifugation, and the resultant supernatants were collected for HPLC (high performance liquid chromatography) analysis.

The results of the dissolution test were presented as the percentage of dissolved pregabalin (%)under different time.

### Results:

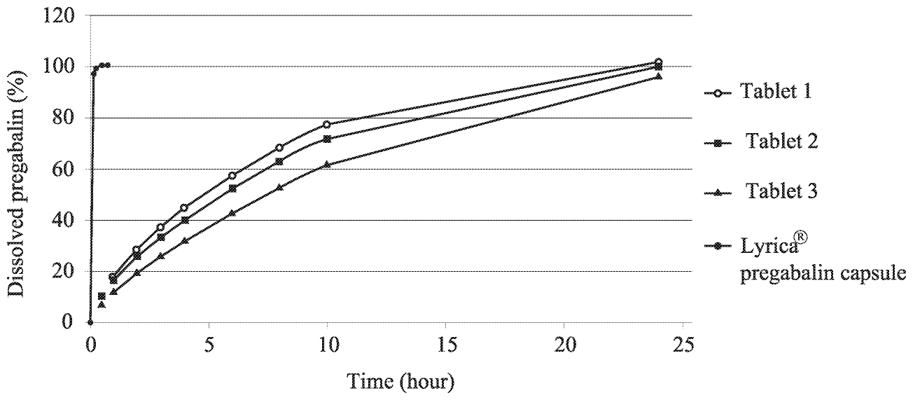

FIG. 1 shows the results of the dissolution test of tablets 1 to 3 of Example 1 and the LYRICA® pregabalin capsule. As shown in FIG. 1, at 0.5 hours after initiation of the dissolution test, the percentage of dissolved pregabalin of the LYRICA® pregabalin capsule was 100%, whereas the percentages of dissolved pregabalin of tablets 1 to 3 were 10.7%, 10.4%, and 7.3%, respectively. In particular, the percentages of dissolved pregabalin of tablets 1 and 2 reached 100% at 24 hours after initiation of the dissolution test, and the percentage of dissolved pregabalin of the tablet 3 reached 100% after more than 24 hours. This result indicates that the sustained-release formulation containing pregabalin of the present disclosure is capable of releasing pregabalin consistently over a 24-hour time period, and hence is suitable for once-daily administration.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A sustained-release formulation comprising, based on the total weight of the sustained-release formulation:
5 wt % to 40 wt % of pregabalin, or a pharmaceutically acceptable salt, solvate or hydrate thereof;
0.1 wt % to 5 wt % of carbomer; and
20 wt % to 60 wt % of polyethylene oxide;
wherein the sustained-release formulation is free from polyvinyl acetate.

2. The sustained-release formulation of claim 1, further comprising, based on the total weight of the sustained-release formulation:
5 wt % to 60 wt % of a diluent;
1 wt % to 10 wt % of a binder;
0.01 wt % to 2 wt % of an antioxidant; and
0.1 wt % to 5 wt % of a lubricant.

3. The sustained-release formulation of claim 2, wherein the diluent is selected from the group consisting of starch, microcrystalline cellulose, anhydrous dicalcium phosphate, dextrose, lactose, sucrose, mannitol, xylitol, sorbitol, and combinations thereof.

4. The sustained-release formulation of claim 2, wherein the binder is selected from the group consisting of polyvinylpyrrolidone (PVP), copovidone, hydroxypropyl cellulose, hydroxypropylmethyl cellulose, hydroxyethyl cellulose, methylcellulose, and combinations thereof.

5. The sustained-release formulation of claim 2, wherein the antioxidant is selected from the group consisting of butylated hydroxytoluene (BHT), butylated hydroxyanisole (BHA), ascorbic acid, propyl gallate, and combinations thereof.

6. The sustained-release formulation of claim 2, wherein the lubricant is selected from the group consisting of stearic acid, magnesium stearate, calcium stearate, zinc stearate, glyceryl monostearate, sodium stearyl fumarate, glyceryl behenate, and combinations thereof.

7. The sustained-release formulation of claim 1, wherein the formulation is in a dosage form for oral administration.
